(19) United States
(12) Patent Application Publication
(10) Pub. No.: US 2024/0023980 A1
(43) Pub. Date: Jan. 25, 2024

(54) INSTRUMENTATION AND METHODS FOR TOTAL ANKLE ARTHROPLASTY

(71) Applicant: Paragon 28, Inc., Englewood, CO (US)

(72) Inventors: Joseph DOGUÉ, Aurora, CO (US); Randy ALLARD, Golden, CO (US); Duncan MELTON, Centennial, CO (US); Zachary Ryan WOODS, Centennial, CO (US)

(73) Assignee: Paragon 28, Inc., Englewood, CO (US)

(21) Appl. No.: 18/480,105

(22) Filed: Oct. 3, 2023

Related U.S. Application Data
(63) Continuation of application No. PCT/US2022/071550, filed on Apr. 5, 2022.
(60) Provisional application No. 63/170,879, filed on Apr. 5, 2021.

(51) Int. Cl. A61B 17/17; A61B 17/15

(52) U.S. Cl. CPC A61B 17/1775; A61B 17/15

(57) ABSTRACT

A surgical guide that includes a front portion and a rear portion that is opposite the front portion, and at least one cut slot extending from the front portion to the rear portion. The at least one cut slot defines a vertical and a horizontal range of motion that extends beyond the lateral dimensions of the at least one cut slot. An alternative surgical guide is disclosed that has a front portion with a front surface and a rear portion that has a rear surface. The surgical guide includes at least one cut slot extending from the front surface to the rear surface which provides fluid communication therebetween. The at least one cut slot has edges defining a vertical and horizontal range of motion for a first depth has bounds defining a horizontal range of motion for a second depth of the at least one cut slot.

### CROSS REFERENCE TO RELATED APPLICATION

This application is a bypass continuation of PCT Application No. PCT/US2022/071550, filed Apr. 5, 2022, and entitled “Instrumentation and Methods for Total Ankle Arthroplasty,” which claims priority benefit under 35 U.S.C. § 119(e) of U.S. Provisional Application No. 63/170,879 filed Apr. 5, 2021, and entitled “Instrumentation and Methods for Total Ankle Arthroplasty,” which are both incorporated herein by reference in their entirety.

The present disclosure relates to surgical guides to be implemented in conjunction with arthroplasty systems including implants, instrumentation, and other devices as well as arthroplasty methodology. The present disclosure relates to podiatric and orthopedic surgical guides (and corresponding arthroplasty systems including implants, instrumentation, methodology, etc.) to be implemented in arthroplasty procedures of the foot and/or ankle. More specifically, but not exclusively, the present disclosure relates to surgical guides to be implemented in conjunction with instruments, implants, devices, systems, assemblies, and methods for performing arthroplasty of the ankle joint.

### BACKGROUND OF THE INVENTION

Many currently available surgical guides as well as corresponding implants, instrumentation, systems, and methods for arthroplasty do not completely address the needs of patients. Additionally, many currently available surgical guides fail to account for properties of ankle and foot anatomy and accordingly can decrease favorability of the outcome for the patient.

### SUMMARY OF THE INVENTION

The present disclosure is directed toward surgical guides for implementation in conjunction with implants, instruments, and methods directed to arthroplasty procedures.

A first aspect of the present disclosure includes a surgical guide. The surgical guide includes a front portion, a rear portion opposite the front portion, and at least one cut slot extending from the front portion to the rear portion. The cut slot defines a vertical and horizontal range of motion, wherein the horizontal range of motion extends beyond lateral dimensions of the surgical guide.

According to one aspect of the present disclosure, the front portion is integral with the rear portion of the surgical guide.

According to one aspect of the present disclosure, the front portion includes a front surface, and the rear portion includes a rear surface.

According to one aspect of the present disclosure, the cut slot is configured to provide fluid communication between the front surface and the rear surface.

According to one aspect of the present disclosure, the surgical guide may include a pair of projections extending laterally from the surgical guide on opposite ends of the at least one cut slot.

According to one aspect of the present disclosure, each of the pair of projections includes at least one through hole.

According to one aspect of the present disclosure, each of the pair of projections includes at least one lobe extending from the protrusion.

According to one aspect of the present disclosure, each of the at least one through holes is disposed at least partially within the at least one lobe of each of the pair of projections.

According to one aspect of the present disclosure, the at least one cut slot includes at least one aperture disposed at least partially within the cut slot and providing fluid communication between the front surface and the rear surface of the surgical guide.

According to one aspect of the present disclosure, the at least one cut slot includes a first cut slot with a first lateral dimension and a second cut slot with a second lateral dimension, wherein the first cut slot is disposed above the second cut slot.

According to one aspect of the present disclosure, the first lateral dimension is equal to the second lateral dimension.

According to one aspect of the present disclosure, at least one of the first cut slot and the second cut slot include at least one aperture disposed at least partially within at least one of the first cut slot and the second cut slot and providing fluid communication between the front surface and the rear surface of the surgical guide.

According to one aspect of the present disclosure, the at least one aperture includes a pair of apertures.

According to one aspect of the present disclosure, the first cut slot includes a first depth and the second cut slot includes a second depth, wherein the first depth is greater than the second depth.

According to one aspect of the present disclosure, the first cut slot is defined in superior and inferior directions for the entirety of the first depth of the first cut slot, and wherein the first cut slot is defined in medial and lateral directions of at least a portion of the first depth of the first cut slot.

According to one aspect of the present disclosure, the second cut slot is defined in superior and inferior directions for the entirety of the second depth of the second cut slot, and wherein the second cut slot is defined in medial and lateral directions of at least a portion of the second depth of the second cut slot.

A second aspect of the present disclosure includes a surgical guide with a front portion having a front surface and a rear portion having a rear surface, with the rear surface opposite the front surface. The surgical guide also includes at least one cut slot extending from the front surface to the rear surface and providing fluid communication therebetween, wherein the at least one cut slot includes bounds defining a vertical and horizontal range of motion for a first depth of the at least one cut slot, and wherein the at least one cut slot includes bounds defining a horizontal range of motion for a second depth of the at least one cut slot.

According to one aspect of the present disclosure, the first cut slot includes a first lateral dimension and a first vertical dimension, with the first cut slot defining a first lateral range of motion for a cutting instrument and the first lateral range of motion extending beyond the lateral-most points of the first cut slot. The second cut slot includes a second lateral dimension and a second vertical dimension. The second cut slot defines a second lateral range of motion for a cutting instrument, wherein the second lateral range of motion extends beyond the lateral-most points of the second cut slot.

A third aspect of the present disclosure includes a surgical guide having a front portion with a front surface and a rear portion with a rear surface. The surgical guide also includes a first cut slot providing fluid communication between the front surface and the rear surface and includes a first depth and a first lateral dimension. The surgical guide also includes a second cut slot providing fluid communication between the front surface and the rear surface and includes a second depth and a second lateral dimension. The first depth may be greater than the second depth and the first lateral dimension may be equal to the second lateral dimension.

According to one aspect of the present disclosure, the first cut slot includes a through hole extending from the front surface to the rear surface, and the second cut slot includes at least one through hole extending from the front surface to the rear surface.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a top view of an exemplary instrument for facilitating implantation of an implant system, in accordance with the present disclosure;

FIG. 2 is a front view of the exemplary instrument of FIG. 1 for facilitating implantation of an implant system, in accordance with the present disclosure;

FIG. 3 is a top perspective view of the exemplary instrument of FIG. 1, in accordance with the present disclosure;

FIG. 4 is a side view of the exemplary instrument of FIG. 1 for facilitating implantation of an implant system, in accordance with the present disclosure;

FIG. 5 is a top view of another exemplary instrument for facilitating implantation of an implant system, in accordance with the present disclosure;

FIG. 6 is a front view of the exemplary instrument of FIG. 5 for facilitating implantation of an implant system, in accordance with the present disclosure;

FIG. 7 is a top perspective view of the exemplary instrument of FIG. 5, in accordance with the present disclosure;

FIG. 8 is a side view of the exemplary instrument of FIG. 5, in accordance with the present disclosure;

FIG. 9 is a top view of another exemplary instrument for facilitating implantation of an implant system, in accordance with the present disclosure;

FIG. 10 is a front view of the exemplary instrument of FIG. 9, in accordance with the present disclosure;

FIG. 11 is a top perspective view of the exemplary instrument of FIG. 9, in accordance with the present disclosure;

FIG. 12 is a side view of the exemplary instrument of FIG. 9 for facilitating implantation of an implant system, in accordance with the present disclosure;

FIG. 13 is a top view of another exemplary instrument for facilitating implantation of an implant system, in accordance with the present disclosure;

FIG. 14 is a front view of the exemplary instrument of FIG. 13, in accordance with the present disclosure;

FIG. 15 is a top perspective view of the exemplary instrument of FIG. 13, in accordance with the present disclosure;

FIG. 16 is a side view of the exemplary instrument of FIG. 13, in accordance with the present disclosure;

FIG. 17 is a top view of another exemplary instrument for facilitating implantation of an implant system, in accordance with the present disclosure;

FIG. 18 is a front view of the exemplary instrument of FIG. 17, in accordance with the present disclosure;

FIG. 19 is a top perspective view of the exemplary instrument of FIG. 17, in accordance with the present disclosure;

FIG. 20 is a side view of the exemplary instrument of FIG. 17, in accordance with the present disclosure;

FIG. 21 is a front perspective view of an exemplary instrument for facilitating alignment of an implant system, in accordance with the present disclosure;

FIG. 22 is an exploded, side cross-sectional view of the exemplary instrument of FIG. 21, in accordance with the present disclosure;

FIG. 23 is a front view of the exemplary instrument of FIG. 21, in accordance with the present disclosure;

FIG. 24 is a side view of the exemplary instrument of FIG. 21, in accordance with the present disclosure;

FIG. 25 is an additional side view of the exemplary instrument of FIG. 21, in accordance with the present disclosure;

FIG. 26 is a perspective view of an additional exemplary instrument for facilitating alignment of an implant system, in accordance with the present disclosure;

FIG. 27 is a side perspective view of the exemplary instrument of FIG. 26, in accordance with the present disclosure;

FIG. 28 is an additional side perspective view of the exemplary instrument of FIG. 26, in accordance with the present disclosure;

FIG. 29 is a rear view of the exemplary instrument of FIG. 26, in accordance with the present disclosure; and

FIG. 30 is a side cross-sectional side view of the exemplary instrument of FIG. 26, in accordance with the present disclosure.

### DETAILED DESCRIPTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; which are hereby incorporated herein by reference in their entireties.

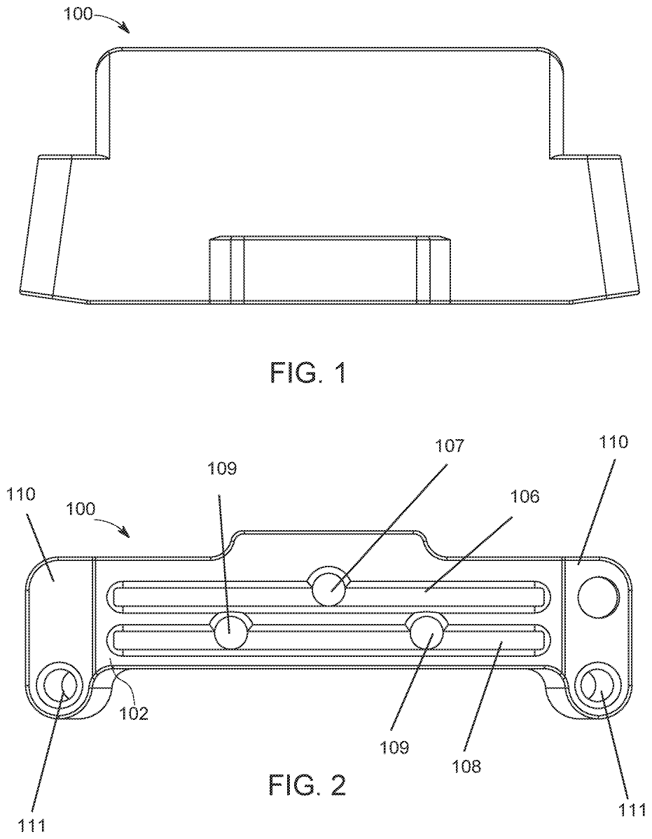

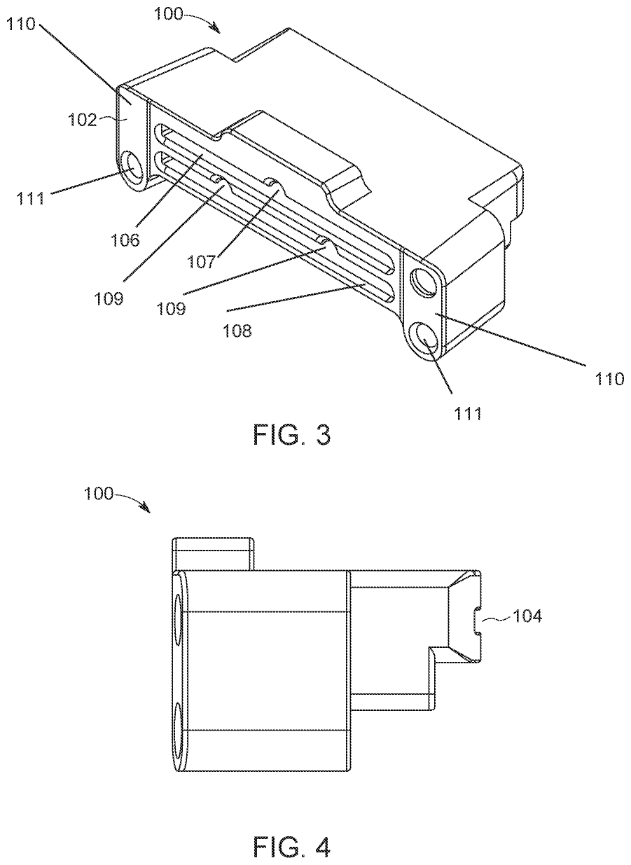

Referring to the drawings, wherein like reference numerals are used to indicate like or analogous components throughout the several views, and with particular reference to FIGS. 1-4, there is illustrated an exemplary embodiment of a surgical guide 100 (e.g., guide, cut guide, saw guide, cut slot guide, surgical guide, resection guide, etc.) for determining, aligning, positioning and/or guiding one or more actions (e.g., cuts/incisions to be made, etc.) to be taken during an ankle arthroplasty procedure.

FIGS. 1-4 illustrate an exemplary instrument, shown as the surgical guide 100, for determining, aligning, positioning and/or guiding one or more cuts to be made in performing total ankle replacement (e.g., ankle arthroplasty) in accordance with the present disclosure. The surgical guide 100 is shown to include a front portion 102, wherein the front portion 102 includes various slots, apertures, bores, and/or other similar features extending from the front portion 102 into and through the surgical guide 100. In some aspects, the slots, apertures and bores of the front portion 102 of the surgical guide 100 may be configured to receive one or more surgical instruments. For example, one or more bores of the front portion 102 may be configured to receive stabilization elements (e.g., k-wires, olive wires, etc.) to releasably couple the surgical guide with a portion of the tibia and/or talus of a patient. In some aspects, the surgical guide 100 may include one or more features configured to facilitate with other surgical instruments and/or equipment (for example, those of the patent applications incorporated by reference herein). Further, one or more of the apertures and/or slots may be configured to receive one or more cutting instruments or other instruments configured to facilitate cutting. For example, the one or more apertures may be configured to receive a drill (e.g., drill bit) or other components. The one or more slots of the front portion 102 may be variously sized to receive one or more cutting instruments, for example a reciprocating saw, into and through the surgical guide 100.

The surgical guide 100 further includes an exit slot 104, where the exit slot 104 may be in fluid communication with one or more of the apertures, bores, and/or slots of the front portion 102. As shown in FIGS. 1-4, the exit slot 104 is shown to be in fluid communication with a cut slot 106 disposed on the front surface of the front portion 102, wherein the cut slot 106 extends through the surgical guide 100 from the front portion 102 to the rear portion. In some aspects, the exit slot 104 may be configured to enable and/or restrict movement of one or more of the aforementioned cutting instruments to a specific range of motion. For example, the exit slot 104 may include lateral boundaries that prevent a cutting instrument from straying outside of a desired range and thus contacting (e.g., cutting, damaging, etc.) anatomical structures other than those positioned within the range of motion permitted by the exit slot 104. In some aspects, the surgical guide 100 may include multiple exit slots 104, where each exit slot 104 corresponds to one or more of the slots disposed on the front portion 102 of the surgical guide 100. For example, the surgical guide 100 may include an exit slot corresponding to a cut slot 108, wherein the cut slot 108 is disposed below the cut slot 106. Further, in some aspects the surgical guide 100 may include two or more of the exit slot 104, wherein one or more of the exit slot 104 may be of various sizes (e.g., width, height, depth, etc.).

The cut slot 106 is shown to be disposed substantially above the cut slot 108, although the cut slots 106, 108 may have alternate configurations in some embodiments. In some aspects, the cut slots 106, 108 may define a range of motion for a cutting instrument received therein in superior/inferior and medial/lateral directions (e.g., such a cutting instrument reaches a boundary of the range of motion when the cutting instrument contacts the medial/lateral and/or inferior/superior edges of the cut slots 106, 108, where the directions medial and lateral are used arbitrarily to indicate opposite side edges/directions of the cut slots 106, 108). In some aspects, the first and second cut slots 106, 108 may define the same range of motion at different heights relative to the surgical guide 100. However, in some aspects, the cut slots 106, 108 may define different ranges of motion for a cutting instrument. In some aspects, the range of motion defined by the cut slots 106, 108 may be influenced by the presence or absence of the exit slot 104 (e.g., the exit slot 104 may allow for additional lateral range of motion of the cutting instrument while the cutting instrument is received in the exit slot 104 as opposed to abutting an edge of the cut slot 108 that may extend through the surgical guide 108 in its entirety). Further, in some aspects, the cut slots 106, 108 may include various depths which further define lateral ranges of motion of the cutting instruments received therein, and which may also be impacted by the presence/absence of the exit slot 104 or another similar/analogous component.

The surgical guide 100 is further shown to include an aperture 107 disposed at least partially within the cut slot 106, where the aperture 107 extends into and through the surgical guide 100 (e.g., the aperture 107 extends from and establishes fluid communication between the front and rear surfaces of the surgical guide 100). As shown, the aperture 107 is disposed at least partially within the cut slot 106 (e.g., at least a portion of the perimeter of the aperture 107 overlaps with the perimeter of the cut slot 106, and at least a portion of the perimeter of the aperture 107 does not overlap with that of the cut slot 106). The surgical guide 100 is further shown to include a pair of apertures 109 disposed within the cut slot 108 where, similar to the aperture 107 in the cut slot 106, the pair of apertures are disposed at least partially within the cut slot 108 (e.g., at least a portion of the perimeter of the apertures 109 overlap with the perimeter of the cut slot 108, and at least a portion of the perimeter of the apertures 109 do not overlap with that of the cut slot 108). As shown, the apertures 109 are spaced apart from one another and from the lateral edges of the cut slot 108 approximately equidistantly (e.g., there is an equal distance between each aperture 109 and the opposite aperture 109 as well as the nearest lateral edge of the cut slot 108).

The surgical guide 100 is further shown to include a pair of projections 110 (e.g., projections, protrusions, etc.) extending laterally from opposite sides of the surgical guide 100. Each of the projections 110 are shown to extend laterally from the front portion 102 of the surgical guide 100 such that each of the projections 110 are positioned adjacent the medial and lateral-most portions of the cut slots 106, 108. As shown, each of the projections 110 are shown to include a through hole 111 disposed in a lobe protruding from a lower portion of the projection and extending from the front portion 102 through the surgical guide 100 to the rear portion so as to establish fluid communication therethrough. In some aspects, each of the through holes 111 may be configured to receive a temporary fixation/stabilization member therein so as to facilitate releasably coupling of the surgical guide 100 with the anatomy of a patient. In some aspects, each of the projections 110 may have a depth that is less than that of the central portions of the surgical guide 100 (e.g., less than that of the cut slot 106, for example). In some aspects, one or more of the projections 110 may include one or more additional through holes that are the same as and/or similar to the through holes 111 disposed variously about the projections 110.

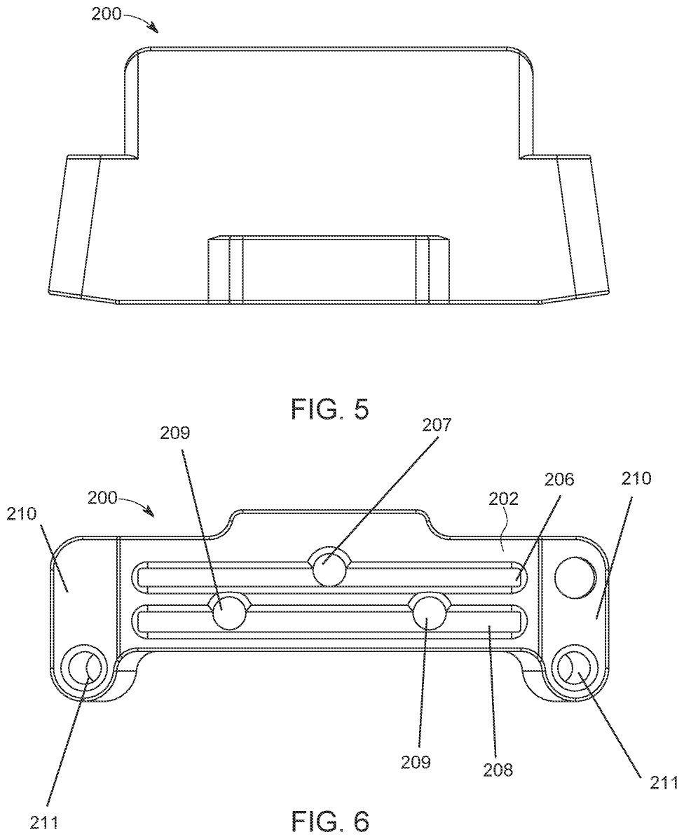

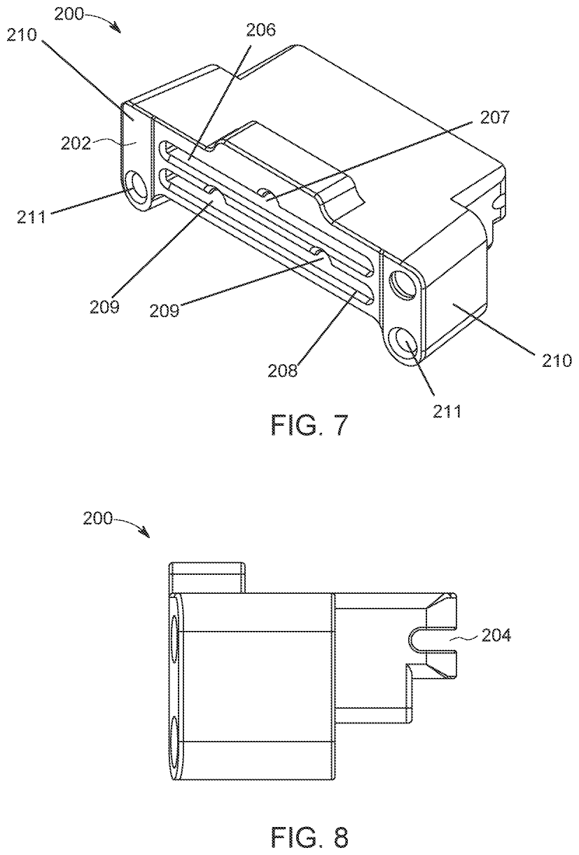

FIGS. 5-8 illustrate an exemplary instrument, shown as the surgical guide 200, for determining, aligning, positioning and/or guiding one or more cuts to be made in performing total ankle replacement (e.g., ankle arthroplasty) in accordance with the present disclosure. The surgical guide 200 is shown to include a front portion 202, wherein the front portion 202 includes various slots, apertures, bores, and/or other similar features extending from the front portion 202 into and through the surgical guide 200. In some aspects, the slots, apertures and bores of the front portion 202 of the surgical guide 200 may be configured to receive one or more surgical instruments. For example, one or more bores of the front portion 202 may be configured to receive stabilization members (e.g., k-wires, olive wires, etc.) to releasably couple the surgical guide with a portion of the tibia and/or talus of a patient. In some aspects, the surgical guide 200 may include one or more features configured to facilitate with other surgical instruments and/or equipment (for example, those of the patent applications incorporated by reference herein). Further, one or more of the apertures and/or slots may be configured to receive one or more cutting instruments or other instruments configured to facilitate cutting. For example, the one or more apertures may be configured to receive a drill (e.g., drill bit) or other components. The one or more slots of the front portion 202 may be variously sized to receive one or more cutting instruments, for example a reciprocating saw, into and through the surgical guide 200.

The surgical guide 200 further includes an exit slot 204, where the exit slot 204 may be in fluid communication with one or more of the apertures, bores, and/or slots of the front portion 202. As shown in FIGS. 5-8, the exit slot 204 is shown to be in fluid communication with a cut slot 206 disposed on the front surface of the front portion 202, wherein the cut slot 206 extends through the surgical guide 200 from the front portion 202 to the rear portion. In some aspects, the exit slot 204 may be configured to enable and/or restrict movement of one or more of the aforementioned cutting instruments to a specific range of motion. For example, the exit slot 204 may include lateral boundaries that prevent a cutting instrument from straying outside of a desired range and thus contacting (e.g., cutting, damaging, etc.) anatomical structures other than those positioned within the range of motion permitted by the exit slot 204. In some aspects, the surgical guide 200 may include multiple exit slots 204, where each exit slot 204 corresponds to one or more of the slots disposed on the front portion 202 of the surgical guide 200. For example, the surgical guide 200 may include an exit slot corresponding to the cut slot 208, wherein the cut slot 208 is disposed below the cut slot 206. Further, in some aspects the surgical guide 200 may include two or more of the exit slot 204, wherein one or more of the exit slot 204 may be of various sizes (e.g., width, height, depth, etc.).

The cut slot 206 is shown to be disposed substantially above the cut slot 208, although the cut slots 206, 208 may have alternate configurations in some embodiments of the surgical guide 200. In some aspects, the cut slots 206, 208 may define a range of motion for a cutting instrument received therein in superior/inferior and medial/lateral directions (e.g., such a cutting instrument reaches a boundary of the range of motion when the cutting instrument contacts the medial/lateral and/or inferior/superior edges of the cut slots 206, 208, where the directions medial and lateral are used arbitrarily to indicate opposite side edges/directions of the cut slots 206, 208). In some aspects, the first and second cut slots 206, 208 may define the same range of motion at different heights relative to the surgical guide 200. However, in some aspects, the cut slots 206, 208 may define different ranges of motion for a cutting instrument. In some aspects, the range of motion defined by the cut slots 206, 208 may be influenced by the presence or absence of the exit slot 204 (e.g., the exit slot 204 may allow for an additional lateral range of motion of the cutting instrument while the cutting instrument is received in the exit slot 204 as opposed to abutting an edge of the cut slot 208 that may extend through the surgical guide 208 in its entirety). Further, in some aspects, the cut slots 206, 208 may include various depths which further define lateral ranges of motion of cutting instruments received therein and which may also be impacted by the presence/absence of the exit slot 204 or another similar/analogous component.

The surgical guide 200 is further shown to include an aperture 207 disposed at least partially within the cut slot 206, wherein the aperture 207 extends into and through the surgical guide 200 (e.g., the aperture 207 extends from and establishes fluid communication between the front and rear surfaces of the surgical guide 200). As shown, the aperture 207 is disposed at least partially within the cut slot 206 (e.g., at least a portion of the perimeter of the aperture 207 overlaps with the perimeter of the cut slot 206, and at least a portion of the perimeter of the aperture 207 does not overlap with that of the cut slot 206). The surgical guide 200 is further shown to include a pair of apertures 209 disposed within the cut slot 208, wherein similar to the aperture 207 in the cut slot 206, the pair of apertures are disposed at least partially within the cut slot 208 (e.g., at least a portion of the perimeter of the apertures 209 overlap with the perimeter of the cut slot 208, and at least a portion of the perimeter of the apertures 209 do not overlap with that of the cut slot 208). As shown, the apertures 209 are spaced from one another and from the lateral edges of the cut slot 208 approximately equidistantly (e.g., there is an equal distance between each aperture 209 and the opposite aperture 209 as well as the nearest lateral edge of the cut slot 208).

The surgical guide 200 is further shown to include a pair of projections 210 (e.g., projections, protrusions, etc.) extending laterally from opposite sides of the surgical guide 200. Each of the projections 210 are shown to extend laterally from the front portion 202 of the surgical guide 200 such that each of the projections 210 are positioned adjacent the medial and lateral-most portions of the cut slots 206, 208. As shown, each of the projections 210 are shown to include a through hole 211 disposed in a lobe protruding from a lower portion of the projection and extending from the front portion 202 through the surgical guide 200 to the rear portion so as to establish fluid communication therethrough. In some aspects, each of the through holes 211 may be configured to receive a temporary fixation/stabilization members therein so as to facilitate releasably coupling of the surgical guide 200 with the anatomy of a patient. In some aspects, each of the projections 210 may have a depth that is less than that of the central portions of the surgical guide 200 (e.g., less than that of the cut slot 206, for example). In some aspects, one or more of the projections 210 may include one or more additional through holes that are the same as and/or similar to the through holes 211 disposed variously about the projections 210.

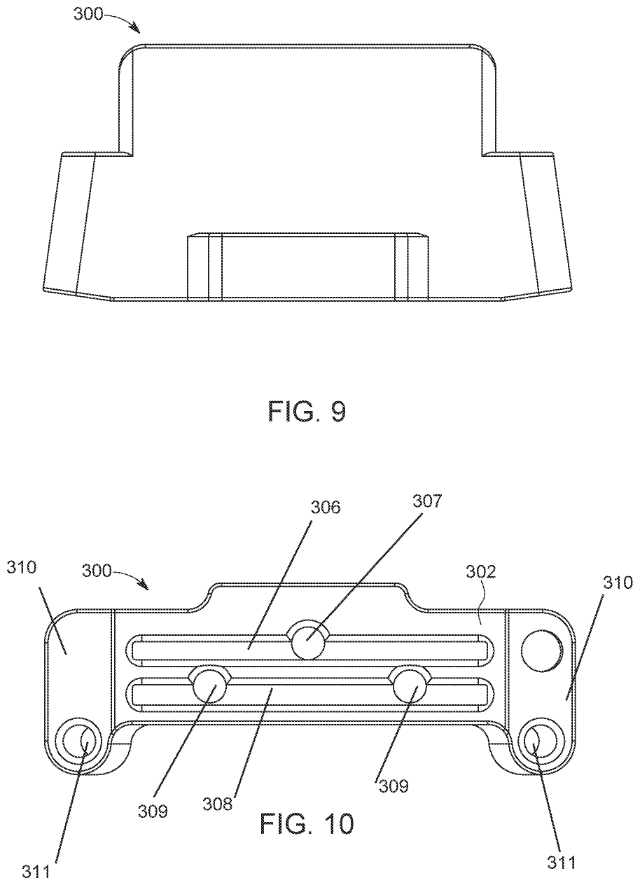

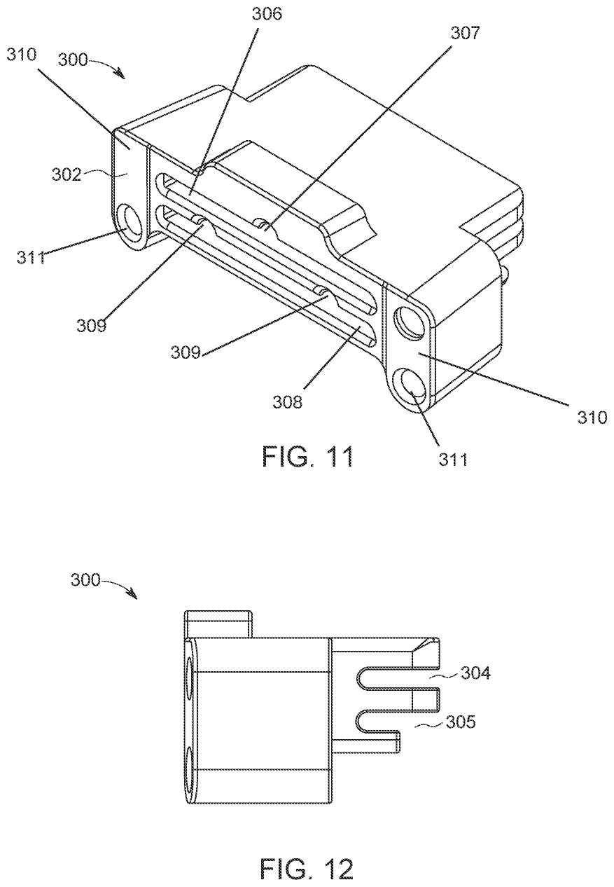

FIGS. 9-12 illustrate an exemplary instrument, shown as the surgical guide 300, for determining, aligning, positioning and/or guiding one or more cuts to be made in performing total ankle replacement (e.g., ankle arthroplasty) in accordance with the present disclosure. The surgical guide 300 is shown to include a front portion 302, wherein the front portion 302 includes various slots, apertures, bores, and/or other similar features extending from the front portion 302 into and through the surgical guide 300. In some aspects, the slots, apertures and bores of the front portion 302 of the surgical guide 300 may be configured to receive one or more surgical instruments. For example, one or more bores of the front portion 302 may be configured to receive stabilization elements (e.g., k-wires, olive wires, etc.) to releasably couple the surgical guide with a portion of the tibia and/or talus of a patient. In some aspects, the surgical guide 300 may include one or more features configured to facilitate with other surgical instruments and/or equipment (for example, those of the patent applications incorporated by reference herein). Further, one or more of the apertures and/or slots may be configured to receive one or more cutting instruments or other instruments configured to facilitate cutting. For example, the one or more apertures may be configured to receive a drill (e.g., drill bit) or other components. The one or more slots of the front portion 302 may be variously sized to receive one or more cutting instruments, for example a reciprocating saw, into and through the surgical guide 300.

The surgical guide 300 further includes a first exit slot 304 and a second exit slot 305, where the exit slots 304, 305 may be in fluid communication with one or more of the apertures, bores, and/or slots of the front portion 302. As shown in FIGS. 9-12, the exit slot 304 is shown to be in fluid communication with a cut slot 306 disposed on the front surface of the front portion 302, wherein the cut slot 306 extends through the surgical guide 300 from the front portion 302 to the rear portion In some aspects, the exit slots 304, 305 may be configured to enable and/or restrict movement of one or more of the aforementioned cutting instruments to a specific range of motion. For example, the exit slot 304 may include lateral boundaries that prevent a cutting instrument from straying outside of a desired range and thus establish a first range of motion (e.g., to prevent contacting, cutting, damaging, etc. anatomical structures other than those positioned within the first range of motion permitted by the exit slot 304). Similarly, the second exit slot 305 may include lateral boundaries that prevent a cutting instrument from straying outside of a desired range, and thus establish a second range of motion. In some aspects, the dimensions and corresponding boundaries of the second exit slot 305 may be different than that of the first exit slot 304 and, accordingly, the second range of motion may be the same as and/or different than the first range of motion. In some aspects, the surgical guide 300 may include multiple exit slots 304 and 305 where each exit slot 304 and/or 305 corresponds to one or more of the slots disposed on the front portion 302 of the surgical guide 300. For example, the surgical guide 300 may include an exit slot corresponding to a cut slot 308, wherein the cut slot 308 is disposed below the cut slot 306. Further, in some aspects the surgical guide 300 may include two or more of the exit slots 304, 305, wherein one or more of the exit slots 304, 306 may be of various sizes (e.g., width, height, depth, etc.).

For example, the cut slot 306 is shown to be disposed substantially above the cut slot 308, although the cut slots 306, 308 may have alternate configurations in some embodiments of the surgical guide 300. In some aspects, the cut slots 306, 308 may define a range of motion for a cutting instrument received therein in superior/inferior and medial/lateral directions (e.g., such a cutting instrument reaches a boundary of the range of motion when the cutting instrument contacts the medial/lateral and/or inferior/superior edges of the cut slots 306, 308, wherein the directions medial and lateral are used arbitrarily to indicate opposite side edges/directions of the cut slots 306, 308). In some aspects, the first and second cut slots 306, 308 may define the same range of motion at different heights relative to the surgical guide 300. However, in some aspects, the cut slots 306, 308 may define different ranges of motion for a cutting instrument. In some aspects, the range of motion defined by the cut slots 306, 308 may be influenced by the presence or absence of the exit slots 304, 305 (e.g., the exit slots 304, 305 may allow for additional lateral range of motion of the cutting instrument while the cutting instrument is received in the exit slots 304, 305 as opposed to abutting an edge of the cut slot 308 that may extend through the surgical guide 308 in its entirety). Further, in some aspects, the cut slots 306, 308 may include various depths which further define lateral ranges of motion of the cutting instruments received therein and which may also be impacted by the presence/absence of the exit slot 304 or another similar/analogous component.

The surgical guide 300 is further shown to include an aperture 307 disposed at least partially within the cut slot 306, where the aperture 307 extends into and through the surgical guide 300 (e.g., the aperture 307 extends from and establishes fluid communication between the front and rear surfaces of the surgical guide 300). As shown, the aperture 307 is disposed at least partially within the cut slot 306 (e.g., at least a portion of the perimeter of the aperture 307 overlaps with the perimeter of the cut slot 306, and at least a portion of the perimeter of the aperture 307 does not overlap with that of the cut slot 306). The surgical guide 300 is further shown to include a pair of apertures 309 disposed within the cut slot 308, wherein similar to the aperture 307 in the cut slot 306, the pair of apertures 309 are disposed at least partially within the cut slot 308 (e.g., at least a portion of the perimeter of the apertures 309 overlap with the perimeter of the cut slot 308, and at least a portion of the perimeter of the apertures 309 do not overlap with that of the cut slot 308). As shown, the apertures 309 are spaced from one another and from the lateral edges of the cut slot 308 approximately equidistantly (e.g., there is an equal distance between each aperture 309 and the opposite aperture 309 as well as the nearest lateral edge of the cut slot 308).

The surgical guide 300 is further shown to include a pair of projections 310 (e.g., projections, protrusions, etc.) extending laterally from the opposite sides of the surgical guide 300. Each of the projections 310 are shown to extend laterally from the front portion 302 of the surgical guide 300 such that each of the projections 310 are positioned adjacent the medial and lateral-most portions of the cut slots 306, 308. As shown, each of the projections 310 are shown to include a through hole 311 disposed in a lobe protruding from a lower portion of the projection and extending from the front portion 302 through the surgical guide 300 to the rear portion so as to establish fluid communication therethrough. In some aspects, each of the through holes 311 may be configured to receive a temporary fixation/stabilization member therein so as to facilitate releasably coupling of the surgical guide 300 with the anatomy of a patient. In some aspects, each of the projections 310 may have a depth that is less than that of the central portions of the surgical guide 300 (e.g., less than that of the cut slot 306, for example). In some aspects, one or more of the projections 310 may include one or more additional through holes that are the same as and/or similar to the through holes 311 disposed variously about the projections 310.

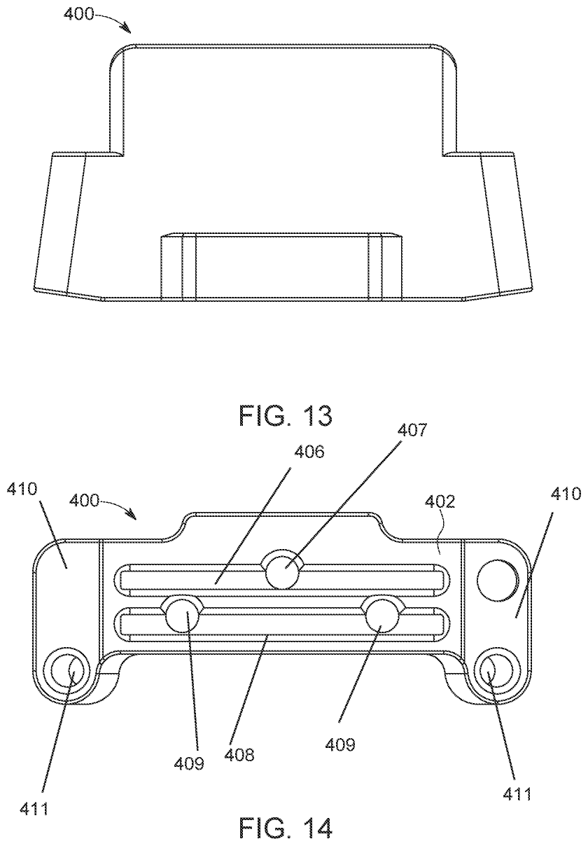

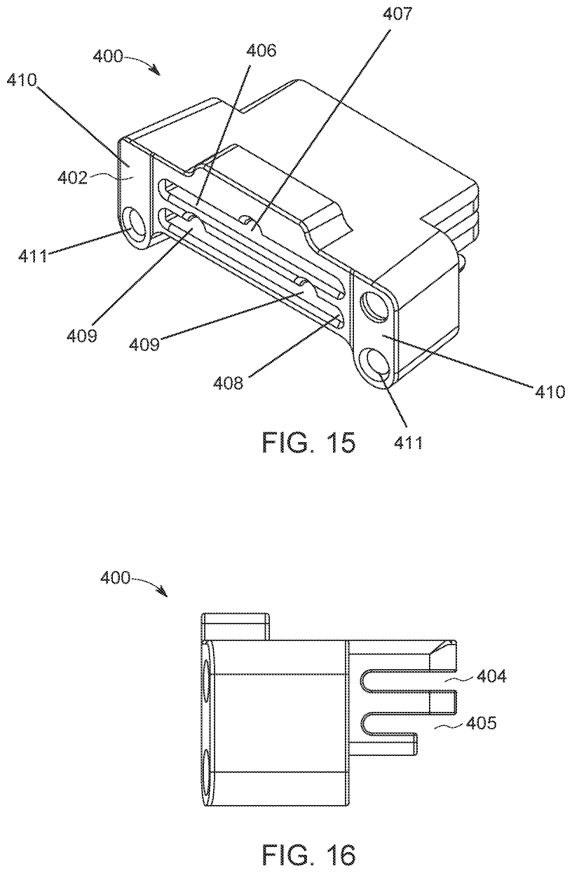

FIGS. 13-16 illustrate an exemplary instrument, shown as the surgical guide 400, for determining, aligning, positioning and/or guiding one or more cuts to be made in performing total ankle replacement (e.g., ankle arthroplasty) in accordance with the present disclosure. The surgical guide 400 is shown to include a front portion 402, wherein the front portion 402 includes various slots, apertures, bores, and/or other similar features extending from the front portion 402 into and through the surgical guide 400. In some aspects, the slots, apertures and bores of the front portion 402 of the surgical guide 400 may be configured to receive one or more surgical instruments. For example, one or more bores of the front portion 402 may be configured to receive stabilization members (e.g., k-wires, olive wires, etc.) to releasably couple the surgical guide with a portion of the tibia and/or talus of a patient. In some aspects, the surgical guide 400 may include one or more features configured to facilitate with other surgical instruments and/or equipment (for example, those of the patent applications incorporated by reference herein). Further, one or more of the apertures and/or slots may be configured to receive one or more cutting instruments or other instruments configured to facilitate cutting. For example, the one or more apertures may be configured to receive a drill (e.g., drill bit) or other components. The one or more slots of the front portion 402 may be variously sized to receive one or more cutting instruments, for example a reciprocating saw, into and through the surgical guide 400.

The surgical guide 400 further includes a first exit slot 404 and a second exit slot 405, wherein the exit slots 404, 405 may be in fluid communication with one or more of the apertures, bores, and/or slots of the front portion 402. As shown in FIGS. 13-16, the exit slot 404 is shown to be in fluid communication with a cut slot 406 disposed on the front surface of the front portion 402, wherein the cut slot 406 extends through the surgical guide 400 from the front portion 402 to the rear portion. In some aspects, the exit slots 404, 405 may be configured to enable and/or restrict movement of one or more of the aforementioned cutting instruments to a specific range of motion. For example, the exit slot 404 may include lateral boundaries that prevent a cutting instrument from straying outside of a desired distance range and thus establish a first range of motion (e.g., to prevent contacting, cutting, damaging, etc. anatomical structures other than those positioned within the first range of motion permitted by the exit slot 404). Similarly, the second exit slot 405 may include lateral boundaries that prevent a cutting instrument from straying outside of a desired distance range and thus establish a second range of motion. In some aspects, the dimensions and corresponding boundaries of the second exit slot 405 may be different than that of the first exit slot 404 and, accordingly, the second range of motion may be the same as and/or different than the first range of motion. In some aspects, the surgical guide 400 may include multiple exit slots 404 and 405, wherein each exit slot 404 and/or 405 corresponds to one or more of the slots disposed on the front portion 402 of the surgical guide 400. For example, the surgical guide 400 may include an exit slot corresponding to a cut slot 408, wherein the cut slot 408 is disposed below the cut slot 406. Further, in some aspects the surgical guide 400 may include two or more of the exit slots 404, 405, wherein one or more of the exit slots 404, 405 may be of various sizes (e.g., width, height, depth, etc.).

The cut slot 406 is shown to be disposed substantially above the cut slot 408, although the cut slots 406, 408 may have alternate configurations in some surgical guide 400 embodiments. In some aspects, the cut slots 406, 408 may define a range of motion for a cutting instrument received therein in superior/inferior and medial/lateral directions (e.g., such a cutting instrument reaches a boundary of the range of motion, wherein the cutting instrument contacts the medial/lateral and/or inferior/superior edges of the cut slots 406, 408, where the directions medial and lateral are used arbitrarily to indicate opposite side edges/directions of the cut slots 406, 408). In some aspects, the first and second cut slots 406, 408 may define the same range of motion at different heights relative to the surgical guide 400. However, in some aspects, the cut slots 406, 408 may define different ranges of motion for a cutting instrument. In some aspects, the range of motion defined by the cut slots 406, 408 may be influenced by the presence or absence of the exit slots 404, 405 (e.g., the exit slots 404, 405 may allow for additional lateral range of motion of the cutting instrument while the cutting instrument is received in the exit slots 404, 405 as opposed to abutting an edge of the cut slot 408 that may extend through the surgical guide 400 in its entirety). Further, in some aspects, the cut slots 406, 408 may include various depths which further define lateral ranges of motion of cutting instruments received therein and which may also be impacted by the presence/absence of the exit slots 404, 405 or another similar/analogous component.

The surgical guide 400 is further shown to include an aperture 407 disposed at least partially within the cut slot 406, where the aperture 407 extends into and through the surgical guide 400 (e.g., the aperture 407 extends from and establishes fluid communication between the front and rear surfaces of the surgical guide 400). As shown, the aperture 407 is disposed at least partially within the cut slot 406 (e.g., at least a portion of the perimeter of the aperture 407 overlaps with the perimeter of the cut slot 406, and at least a portion of the perimeter of the aperture 407 does not overlap with that of the cut slot 406). The surgical guide 400 is further shown to include a pair of apertures 409 disposed within the cut slot 408, wherein, similar to the aperture 407 in the cut slot 406, the pair of apertures are disposed at least partially within the cut slot 408 (e.g., at least a portion of the perimeter of the apertures 409 overlap with the perimeter of the cut slot 408, and at least a portion of the perimeter of the apertures 409 do not overlap with that of the cut slot 408). As shown, the apertures 409 are spaced apart from one another and from the lateral edges of the cut slot 408 approximately equidistantly (e.g., there is an equal distance between each aperture 409 and the opposite aperture 409 as well as the nearest lateral edge of the cut slot 408).

The surgical guide 400 is further shown to include a pair of projections 410 (e.g., projections, protrusions, etc.) extending laterally from opposite sides of the surgical guide 400. Each of the projections 410 are shown to extend laterally from the front portion 402 of the surgical guide 400 such that each of the projections 410 are positioned adjacent the medial and lateral-most portions of the cut slots 406, 408. As shown, each of the projections 410 are shown to include a through hole 411 disposed in a lobe protruding from a lower portion of the projection and extending from the front portion 402 through the surgical guide 400 to the rear portion so as to establish fluid communication therethrough. In some aspects, each of the through holes 411 may be configured to receive a temporary fixation/stabilization member therein so as to facilitate the releasably coupling of the surgical guide 400 with the anatomy of a patient. In some aspects, each of the projections 410 may have a depth that is less than that of the central portions of the surgical guide 400 (e.g., less than that of the cut slot 406, for example). In some aspects, one or more of the projections 410 may include one or more additional through holes that are the same as and/or similar to the through holes 411 disposed variously about the projections 410.

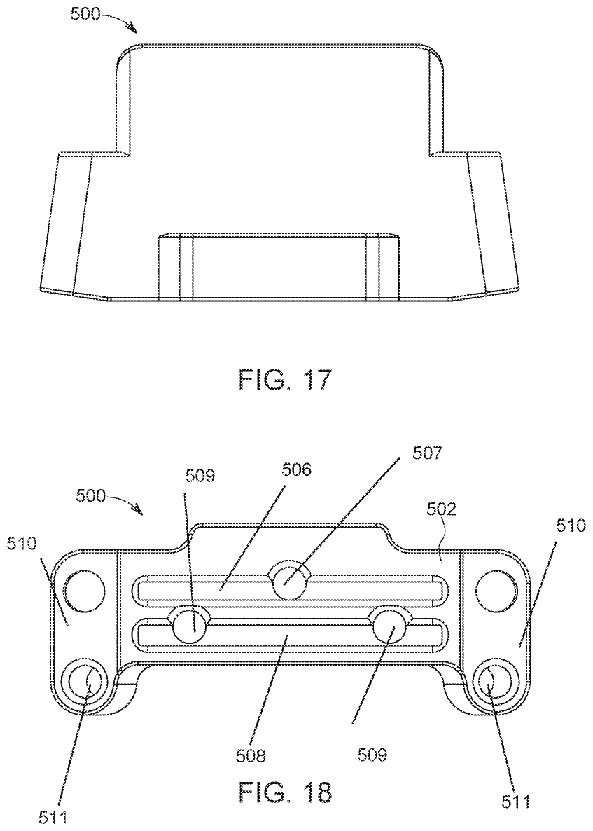

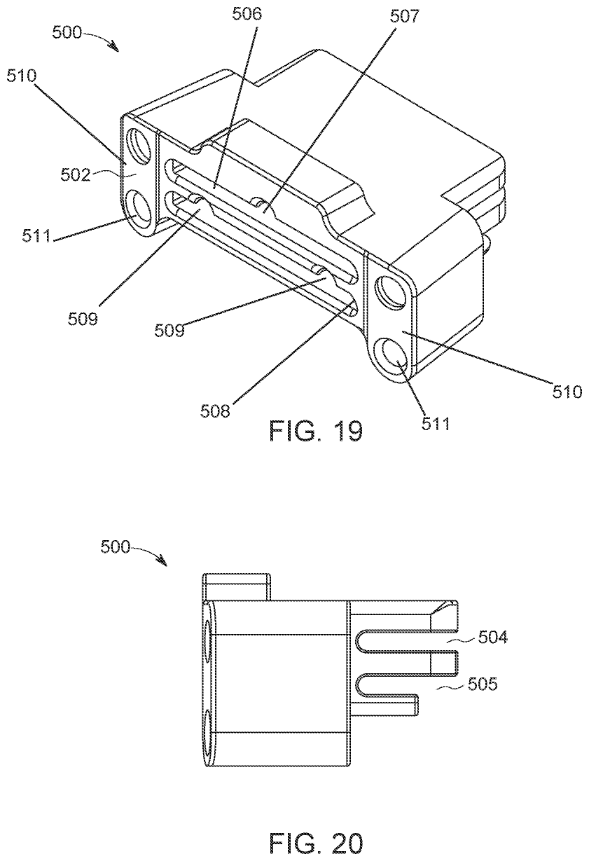

FIGS. 17-20 illustrate an exemplary instrument, shown as the surgical guide 500, for determining, aligning, positioning and/or guiding one or more cuts to be made in performing total ankle replacement (e.g., ankle arthroplasty) in accordance with the present disclosure. The surgical guide 500 is shown to include a front portion 502, wherein the front portion 502 includes various slots, apertures, bores, and/or other similar features extending from the front portion 502 into and through the surgical guide 500. In some aspects, the slots, apertures and bores of the front portion 502 of the surgical guide 500 may be configured to receive one or more surgical instruments. For example, one or more bores of the front portion 502 may be configured to receive stabilization members (e.g., k-wires, olive wires, etc.) to releasably couple the surgical guide 500 with a portion of the tibia and/or talus of a patient. In some aspects, the surgical guide 500 may include one or more features configured to facilitate with other surgical instruments and/or equipment (for example, those of the patent applications incorporated by reference herein). Further, one or more of the apertures and/or slots may be configured to receive one or more cutting instruments or other instruments configured to facilitate cutting. For example, the one or more apertures may be configured to receive a drill (e.g., drill bit) or other components. The one or more slots of the front portion 502 may be variously sized to receive one or more cutting instruments, for example a reciprocating saw, into and through the surgical guide 500.

The surgical guide 500 further includes a first exit slot 504 and a second exit slot 505, where the exit slots 504, 505 may be in fluid communication with one or more of the apertures, bores, and/or slots of the front portion 502. As shown in FIGS. 17-20, the exit slot 504 is shown to be in fluid communication with a cut slot 506 disposed on the front surface of the front portion 502, wherein the cut slot 506 extends through the surgical guide 500 from the front portion 502 to the rear portion. In some aspects, the exit slots 504, 505 may be configured to enable and/or restrict movement of one or more of the aforementioned cutting instruments to a specific range of motion. For example, the exit slot 504 may include lateral boundaries that prevent a cutting instrument from straying outside of a desired distance range and thus establish a first range of motion (e.g., to prevent contacting, cutting, damaging, etc. anatomical structures other than those positioned within the first range of motion permitted by the exit slot 504). Similarly, the second exit slot 505 may include lateral boundaries that prevent a cutting instrument from straying outside of a desired distance range and thus establish a second range of motion. In some aspects, the dimensions and corresponding boundaries of the second exit slot 505 may be different than that of the first exit slot 504 and, accordingly, the second range of motion may be the same as and/or different than the first range of motion. In some aspects, the surgical guide 500 may include multiple exit slots 504 and 505, wherein each exit slot 504 and/or 505 corresponds to one or more of the slots disposed on the front portion 502 of the surgical guide 500. For example, the surgical guide 500 may include an exit slot corresponding to a cut slot 508, wherein the cut slot 508 is disposed below the cut slot 506. Further, in some aspects the surgical guide 500 may include two or more of the exit slots 504, 505, wherein one or more of the exit slot 504 may be of various sizes (e.g., width, height, depth, etc.).

The cut slot 506 is shown to be disposed substantially above the cut slot 508, although the cut slots 506, 508 may have alternate configurations in some embodiments. In some aspects, the cut slots 506, 508 may define a range of motion for a cutting instrument received therein in superior/inferior and medial/lateral directions (e.g., such a cutting instrument reaches a boundary of the range of motion, wherein the cutting instrument contacts the medial/lateral and/or inferior/superior edges of the cut slots 506, 508, wherein the directions medial and lateral are used arbitrarily to indicate opposite side edges/directions of the cut slots 506, 508). In some aspects, the first and second cut slots 506, 508 may define the same range of motion at different heights relative to the surgical guide 500. However, in some aspects, the cut slots 506, 508 may define different ranges of motion for a cutting instrument. In some aspects, the range of motion defined by the cut slots 506, 508 may be influenced by the presence or absence of the exit slots 504, 505 (e.g., the exit slots 504, 505 may allow for additional lateral range of motion of the cutting instrument while the cutting instrument is received in the exit slots 504, 505 as opposed to abutting an edge of the cut slot 508 that may extend through the surgical guide 508 in its entirety). Further, in some aspects, the cut slots 506, 508 may include various depths which further define lateral ranges of motion of cutting instruments received therein and which may also be impacted by the presence/absence of the exit slots 504, 505 or another similar/analogous component.

The surgical guide 500 is further shown to include an aperture 507 disposed at least partially within the cut slot 506, wherein the aperture 507 extends into and through the surgical guide 500 (e.g., the aperture 507 extends from and establishes fluid communication between the front and rear surfaces of the surgical guide 500). As shown, the aperture 507 is disposed at least partially within the cut slot 506 (e.g., at least a portion of the perimeter of the aperture 507 overlaps with the perimeter of the cut slot 506, and at least a portion of the perimeter of the aperture 507 does not overlap with that of the cut slot 506). The surgical guide 500 is further shown to include a pair of apertures 509 disposed within the cut slot 508, wherein similar to the aperture 507 in the cut slot 506, the pair of apertures are disposed at least partially within the cut slot 508 (e.g., at least a portion of the perimeter of the apertures 509 overlap with the perimeter of the cut slot 508, and at least a portion of the perimeter of the apertures 509 do not overlap with that of the cut slot 508). As shown, the apertures 509 are spaced apart from one another and from the lateral edges of the cut slot 508 approximately equidistantly (e.g., there is an equal distance between each aperture 509 and the opposite aperture 509 as well as the nearest lateral edge of the cut slot 508).

The surgical guide 500 is further shown to include a pair of projections 510 (e.g., projections, protrusions, etc.) extending laterally from opposite sides of the surgical guide 500. Each of the projections 510 are shown to extend laterally from the front portion 502 of the surgical guide 500 such that each of the projections 510 are positioned adjacent the medial and lateral-most portions of the cut slots 506, 508. As shown, each of the projections 510 are shown to include a through hole 511 disposed in a lobe protruding from a lower portion of the projection and extending from the front portion 502 through the surgical guide 500 to the rear portion so as to establish fluid communication therethrough. In some aspects, each of the through holes 511 may be configured to receive a temporary fixation/stabilization member therein so as to facilitate the releasably coupling of the surgical guide 500 with the anatomy of a patient. In some aspects, each of the projections 510 may have a depth that is less than that of the central portions of the surgical guide 500 (e.g., less than that of the cut slot 506, for example). In some aspects, one or more of the projections 510 may include one or more additional through holes that are the same as and/or similar to the through holes 511 disposed variously about the projections 510.

It should be understood to one skilled in the art that the surgical guides 100, 200, 300, 400, and 500 may be configured in various sizes so as to accommodate various instruments and/or account for various anatomies of patients. For example, the surgical guide 100 may be of a first size, while the surgical guide 500 may be of a second size. In some aspects, the first size may be larger than the second size and thus account for different anatomical features and/or sized thereof of a patient. Similarly, a physician may implement one or more of the surgical guides in an arthroplasty procedure (e.g., to determine the appropriate size surgical guide for the procedure). Further, one or more of the surgical guides shown and described herein may define one or more ranges of motion (e.g., for a cutting instrument inserted within the one or more slots), wherein the range(s) of motion extend beyond the lateral dimensions of the surgical guides. For example, the surgical guide may include recessed or lateral portions thereof (e.g. “early exit features”) configured to receive at least a portion of a cutting instrument within/throughout a lateral/horizontal a range of motion.

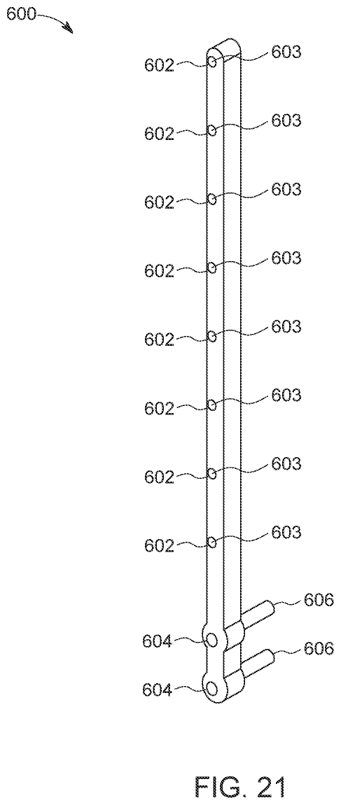

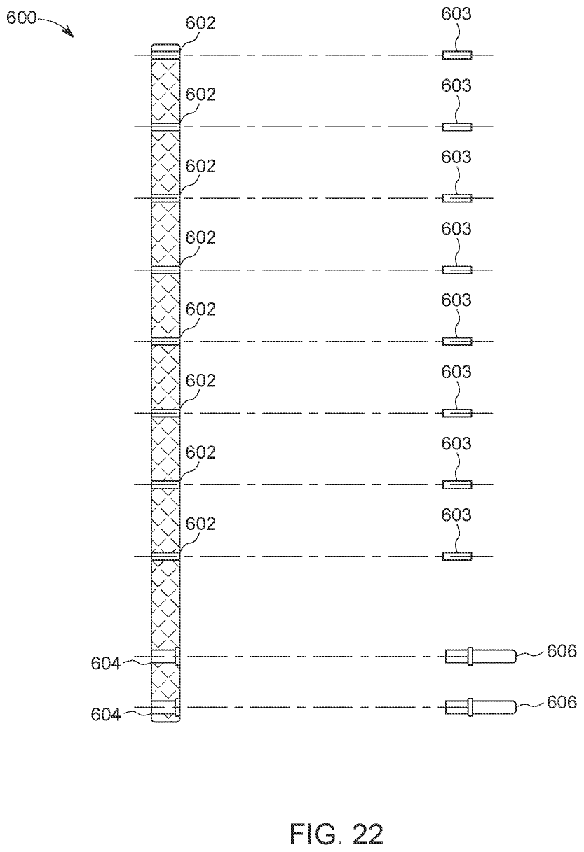

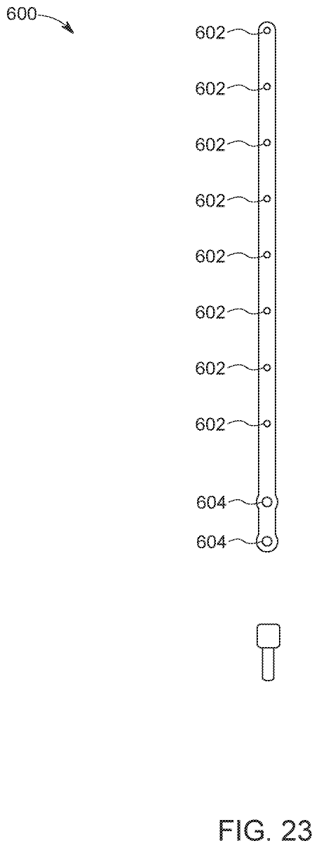

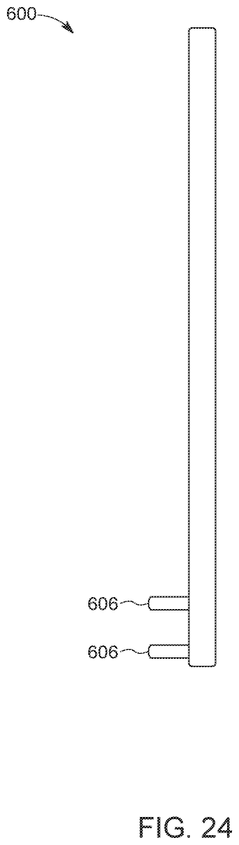

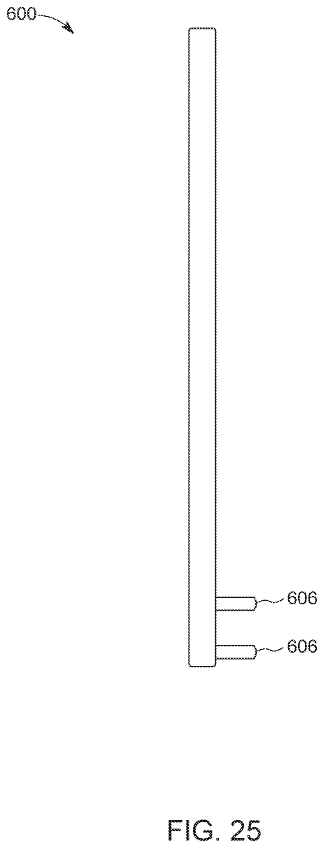

FIGS. 21-25 illustrate an exemplary embodiment of an alignment guide 600, for determining, aligning, and/or positioning one or more elements or components associated with total ankle arthroplasty. The alignment guide 600 may be configured to be implemented in conjunction with one or more components of a total ankle arthroplasty system, for example those discussed herein as well as those of pending patent applications incorporated by reference herein. As shown, the alignment guide 600 includes a plurality of bores 602 configured along a length thereof. The bores 600 are shown to extend through the alignment guide 600 such that fluid communication may be established therethrough. In some aspects, the bores 602 may be spaced at consistent intervals along the length of at least a portion of the alignment guide 600. The alignment guide 600 may be configured to couple with one or more other surgical instruments such that the guide 600 facilitates achieving proper varus-valgus alignment for one or more steps in an arthroplasty procedures and/or instruments and/or implant components. The bores 602 are shown to accommodate pegs 603, wherein the pegs 603 may be configured to be received therein by the bores 602 of the alignment guide 600 so as to releasably couple the pegs 603 with the bores 602. The alignment guide 600 further includes a pair of apertures 604 disposed adjacent to an end of the alignment guide 600 opposite the bores 602. In some aspects, the alignment guide 600 may include a single aperture 604 and/or more than two apertures 604. The apertures 604 are shown to accommodate insertion elements 606 at least partially therein, wherein the insertion elements 606 may be configured to be received therein by the apertures 604 of the alignment guide 600 so as to releasably couple the insertion elements 606 with the apertures 604. In some aspects, the insertion elements 606 may be configured to facilitate coupling of the alignment guide 600 with one or more other components.

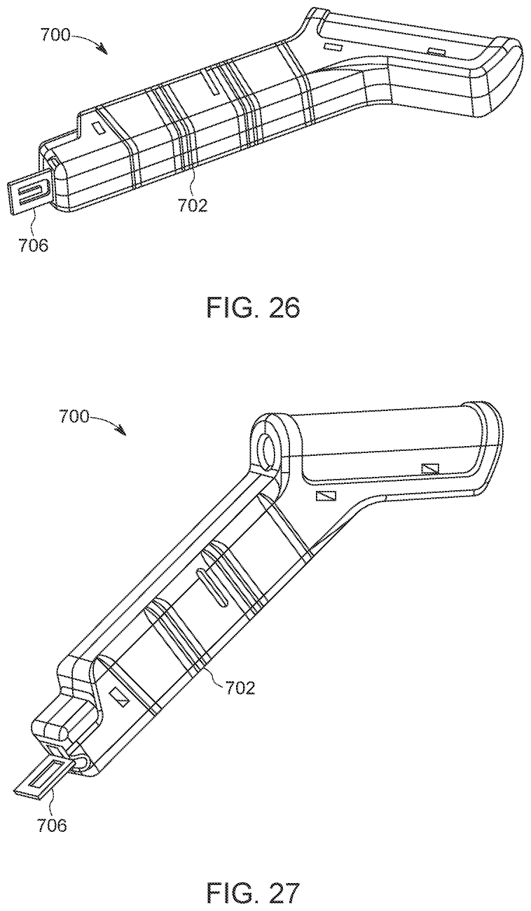

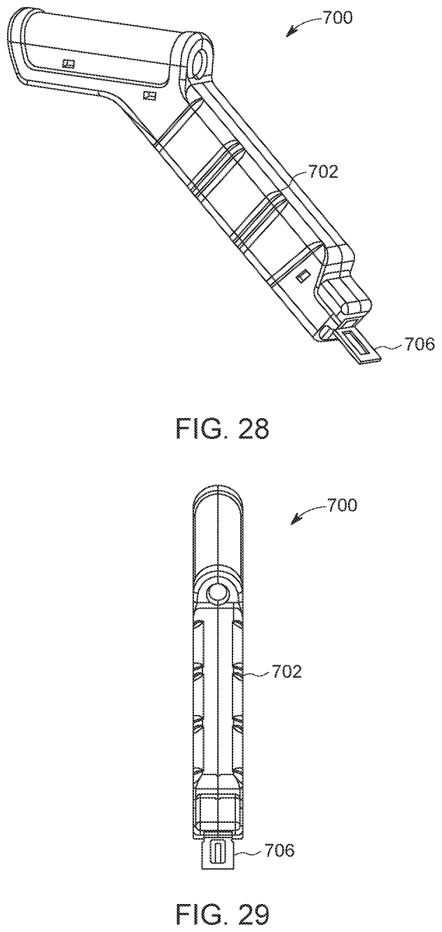

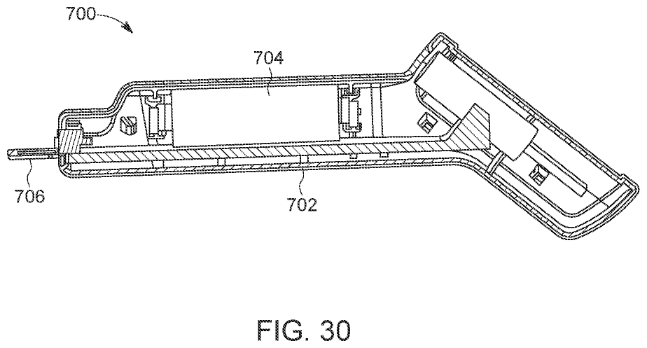

FIGS. 26-30 illustrate an exemplary embodiment of an alignment guide 700, for determining, aligning, and/or positioning one or more elements or components associated with total ankle arthroplasty. As an example, the alignment guide 700 may be a laser alignment guide (as shown), and may be configured to releasably couple with one or more surgical components implemented in a total ankle arthroplasty procedure. Further, the alignment guide (shown as a laser alignment guide) may be configured to couple adjacent a distal portion of the tibia of a patient and, by projecting a laser, facilitate alignment of one or more arthroplasty implant components and/or procedural steps at or near the distal portion of the tibia with the proximal portion of the tibia. The alignment guide 700 is shown to include a housing 702, wherein the housing 702 may include one or more textures or other elements on a surface thereof corresponding to ergonomics of a hand (e.g., of a physician implementing the alignment guide 700 in a procedure). As shown for example, the housing 702 includes multiple ribs positioned along a length thereof, but may include alternate textures (e.g., rubberized coating, tactile patterns, projections, depressions, etc.). The alignment guide 700 also includes a projection 706 arranged on an end thereof, wherein the projection 706 may be configured to facilitate releasable coupling with one or more other surgical instrument components implemented or used in performing a total ankle arthroplasty. As shown in FIG. 30, the alignment guide includes a casing 704 disposed inside the housing 702. The casing 704 is configured to retain a power component (e.g., a battery) therein such that the laser of the alignment guide may be powered by the power element. In some aspects, the casing 704 may also include one or more connection points to establish an electrical connection between a power element within the casing 706 and the laser of the alignment guide 700.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A surgical guide, comprising:
a front portion;
a rear portion opposite the front portion; and
at least one cut slot extending from the front portion to the rear portion, wherein the at least one cut slot defines a vertical and horizontal range of motion, and wherein the horizontal range of motion extends beyond at least one lateral dimension of the at least one cut slot.

2. The surgical guide of claim 1, wherein the front portion is integral with the rear portion of the surgical guide.

3. The surgical guide of claim 1, wherein the front portion comprises a front surface and the rear portion comprises a rear surface.

4. The surgical guide of claim 3, wherein the cut slot is configured to provide fluid communication between the front surface and the rear surface. The surgical guide of claim 3, further comprising a pair of projections extending laterally from the surgical guide on opposite ends of the at least one cut slot.

6. The surgical guide of claim 5, wherein each of the pair of projections comprises at least one through hole.

7. The surgical guide of claim 6, wherein each of the pair of projections comprises at least one lobe extending from the protrusion.

8. The surgical guide of claim 7, wherein each of the at least one through holes is disposed at least partially within the at least one lobe of each of the pair of projections.

9. The surgical guide of claim 3, wherein the at least one cut slot comprises at least one aperture disposed at least partially within the cut slot and providing fluid communication between the front surface and the rear surface of the surgical guide. The surgical guide of claim 3, wherein the at least one cut slot comprises a first cut slot comprising a first lateral dimension and a second cut slot comprising a second lateral dimension, wherein the first cut slot is disposed above the second cut slot.

11. The surgical guide of claim 10, wherein the first lateral dimension is equal to the second lateral dimension.

12. The surgical guide of claim 10, wherein at least one of the first cut slot and the second cut slot comprise at least one aperture disposed at least partially within at least one of the first cut slot and the second cut slot and providing fluid communication between the front surface and the rear surface of the surgical guide.

13. The surgical guide of claim 12, wherein the at least one aperture comprises a pair of apertures.

14. The surgical guide of claim 11, wherein the first cut slot comprises a first depth and the second cut slot comprises a second depth, wherein the first depth is greater than the second depth.

15. The surgical guide of claim 14, wherein the first cut slot is defined in superior and inferior directions for the entirety of the first depth of the first cut slot, and wherein the first cut slot is defined in medial and lateral directions of at least a portion of the first depth of the first cut slot.

16. The surgical guide of claim 14, wherein the second cut slot is defined in superior and inferior directions for the entirety of the second depth of the second cut slot, and wherein the second cut slot is defined in medial and lateral directions of at least a portion of the second depth of the second cut slot.

17. A surgical guide, comprising:
a front portion comprising a front surface;
a rear portion opposite the front portion and comprising a rear surface opposite the front surface; and
at least one cut slot extending from the front surface to the rear surface and providing fluid communication therebetween, wherein the at least one cut slot comprises bounds defining a vertical and horizontal range of motion for a first depth of the at least one cut slot, and wherein the at least one cut slot comprises bounds defining a horizontal range of motion for a second depth of the at least one cut slot.

18. The surgical guide of claim 17, wherein the at least one cut slot comprises:
a first cut slot, comprising:
a first lateral dimension;
a first vertical dimension; and
wherein the first cut slot defines a first lateral range of motion for a cutting instrument, wherein the first lateral range of motion extends beyond the lateral-most edge of the first cut slot; and
a second cut slot disposed below the first cut slot and comprising:
a second lateral dimension;
a second vertical dimension; and
wherein the second cut slot defines a second lateral range of motion for a cutting instrument, wherein the second lateral range of motion extends beyond the lateral-most edge of the second cut slot.

19. A surgical guide, comprising:
a front portion having a front surface;
a rear portion opposite the front portion and comprising a rear surface opposite the front surface;
a first cut slot providing fluid communication between the front surface and the rear surface, comprising:
a first depth; and
a first lateral dimension; and
a second cut slot providing fluid communication between the front surface and the rear surface, comprising:
a second depth; and
a second lateral dimension;
wherein the first depth is greater than the second depth and the first lateral dimension is equal to the second lateral dimension.

20. The surgical guide of claim 19, wherein the first cut slot comprises a through hole extending from the front surface to the rear surface, and wherein the second cut slot comprises at least one through hole extending from the front surface to the rear surface.
